(19) United States
(12) Patent Application Publication
(10) Pub. No.: US 2007/0255252 A1
(43) Pub. Date: Nov. 1, 2007

(54) Embolectomy Catheter

(76) Inventor: Bharat A. Mehta, Bloomfield, MI (US)

Correspondence Address: Kenneth I. Kohn;KOHN & ASSOCIATES, PLLC, 30500 Northwestern Highway, Suite 410, Farmington Hills, MI, 48334, US

(21) Appl. No.: 10/575,034

(22) PCT Filed: Oct. 7, 2004
(86) PCT No.: PCT/US04/33049

§ 371 Date: Mar. 15, 2007

Related U.S. Application Data
(60) Provisional application No. 60/509,377, filed on Oct. 7, 2003.

(51) Int. Cl. A61M 25/01

(52) U.S. Cl. 604/508

(57) ABSTRACT

A grooved embolectomy catheter (10) having lumen including a grooved insertion end (22). A method of treating a thrombus in an individual in need of treatment by inserting the above catheter into an individual, at a location in need of treatment, and rotating the catheter within the individual at the location in need of treatment, thereby breaking apart the thrombus is provided.

### BACKGROUND OF THE INVENTION

1. Field of the Invention

The present invention generally relates to medical devices. More specifically, the present invention relates to an embolectomy catheter.

2. Description of Related Art

Various types of thromboembolic disorders, such as stroke, pulmonary embolism, peripheral thrombosis, atherosclerosis, and the like, are known to occur in human beings and other mammals. Such thromboembolic disorders are typically characterized by the presence of a thromboembolus (i.e., a viscoelastic blood clot comprised of platelets, fibrinogen and other clotting proteins). A thromboembolus (hereinafter “thrombus”) is a clot of blood formed within a blood vessel and remains attached to its place of origin. An embolism is the obstruction of a blood vessel by a foreign or abnormal particle. The occasion of such a thrombosis or embolism within hospitalized patients is one of the leading causes of death.

In cases where the thromboembolism is located in a vein, the obstruction created by the thromboembolus may give rise to a condition of blood stasis, with the development of a condition known as thrombophlebitis within the vein. Moreover, peripheral venous embolisms may migrate to other areas of the body where even more serious effects can result. For example, the majority of pulmonary embolisms are caused by emboli that originate in the peripheral venous system and subsequently migrate through the venous vasculature and become lodged with the lung.

When the thromboembolus is located within an artery, the normal flow of arterial blood may be blocked or disrupted, and tissue ischemia (lack of available oxygen and nutrients required by the tissue) may develop. In such cases, if the thromboembolism is not relieved, the ischemic tissue may become infarcted (i.e., necrotic). Depending on the type and location of the arterial thromboembolus, such tissue infarction can result in death and amputation of a limb, myocardial infarction, or stroke. Notably, strokes caused by thromboemboli that become lodged in the small blood vessels of the brain continue to be a leading cause of death and disability, throughout the world.

In modern medical practice, thromboembolic disorders are typically treated by one or more of the following treatment modalities: a) pharmacologic treatment wherein thrombolytic agents (e.g., streptokinase, urokinase, tissue plasminogen activator (TPA)) and/or anticoagulant drugs (e.g., heparin, warfarin) are administered in an effort to dissolve and prevent further growth of the clot; b) open surgical procedures (e.g., surgical embolectomy or clot removal) wherein an incision is made in the blood vessel in which the clot is lodged and the clot is removed through such incision-sometimes with the aid of a balloon-tipped catheter (e.g., a “Fogarty Catheter”) that is passed through the incision and into the lumen of the blood vessel where its balloon is inflated and used to extract the clot out of the incision; and, c) transluminal catheter-based interventional procedures wherein a clot removing/disrupting catheter (e.g., a suction-type catheter having a suction tip, clot-capturing type catheter having a clot-capturing receptacle (e.g., a basket, coil, hook, etc.), or clot-disrupting catheter having a clot disrupting apparatus (e.g., an ultrasound probe or laser)) is percutaneously inserted and advanced through the patient's vasculature to a location adjacent the clot. The suction tip, clot-capturing receptacle, or clot-disrupting apparatus is used to aspirate, capture and remove, disrupt, or ablate the offending clot.

Each of the above-listed treatment modalities has advantages and disadvantages. For example, pharmacologic treatment has the advantage of being non-invasive and is often effective in lysing or dissolving the clot. However, the thrombolytic and/or anticoagulant drugs used in these pharmacologic treatments can cause side effects such as bleeding or hemorrhage. Also, in cases where time is of the essence, such as cases where an arterial thromboembolism is causing severe tissue ischemia (e.g., an evolving stroke or an evolving myocardial infarction), the time required for the thrombolytic drugs to fully lyse or dissolve the blood clot and restore arterial blood flow may be too long to avoid or minimize the impending infarction.

Open surgical thrombus-removing procedures can, in many cases, be used to rapidly remove clots from the lumens of blood vessels, but such open surgical procedures are notoriously invasive, often requiring general anesthesia. Also, the use of such open surgical procedures is generally limited to blood vessels that are located in surgically accessible areas of the body. For example, many patients suffer strokes due to the lodging of blood clots in small arteries located in surgically inaccessible areas of their brains and, thus, are not candidates for open surgical treatment.

Transluminal, catheter-based interventional procedures are minimally invasive. Such procedures can often be performed without general anesthesia and can be used to rapidly remove a clot from the lumen of a blood vessel. However, such catheter-based interventional procedures are highly operator-skill-dependent and can be difficult or impossible to perform in small or tortuous blood vessels. Thus, patients who suffer strokes due to the presence of clots in the small, tortuous arteries of their brains may not presently be candidates for catheter-based, transluminal removal of the clot, due to the small size and tortuosity of the arteries in which their clots are located.

Additionally, none of the prior art transluminally deployable clot capturing type of catheters are believed to be of optimal design for use in the small blood vessels of the brain because they are: a) not equipped with appropriate guidewire passage lumens to allow them to be passed over previously inserted, small-diameter (e.g., 0.006-0.018 inch) guidewires; b) they are not adapted for rapid exchange over a guidewire of standard length (e.g., a guidewire which is less than twice the length of the catheter); and c) the clot capturing receptacles of these catheters are not optimally constructed and configured for removal of clots from very small blood vessels as are typically found in the brain.

Examples of transluminally deployable clot-capturing type embolectomy catheters of the prior art include those described in U.S. Pat. No. 4,706,671, to Weinrib, U.S. Pat. No. 4,873,978, to Ginsburg, U.S. Pat. No. 5,011,488, to Ginsburg, and PCT International Patent Publication No. WO 97/27808, to Wensel, et al. However, for the reasons stated above and/or other reasons, none of these prior art embolectomy catheters are designed for treating ischemic stroke.

Thus, there exists a need for the development of new embolectomy catheters that are constructed to rapidly and selectively remove blood clots or other matter from small, delicate blood vessels of the brain, so as to provide an effective treatment for evolving strokes and other thromboembolic disorders.

### SUMMARY OF THE INVENTION

According to the present invention, there is provided a grooved embolectomy catheter having an insertion end and an opposite end opposite the insertion end. A method of treating a thrombus in an individual in need of treatment by inserting the above catheter into an individual, at a location in need of treatment, and rotating the catheter within the individual at the location in need of treatment, thereby breaking apart the thrombus is provided.

### DESCRIPTION OF THE DRAWINGS

Other advantages of the present invention are readily appreciated as the same becomes better understood by reference to the following detailed description, when considered in connection with the accompanying drawings wherein:

FIG. 1 is a side view broken away of the catheter of the present invention; and

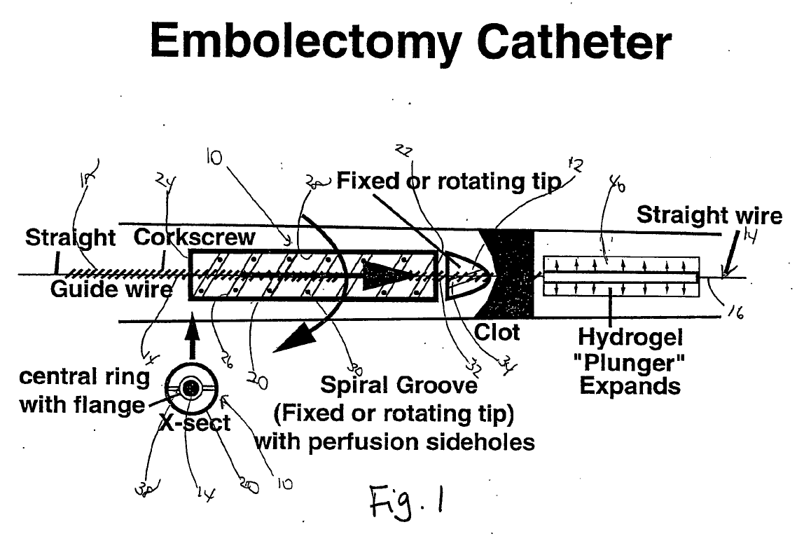

FIGS. 2A through E show several embodiments of the catheter of the present invention.

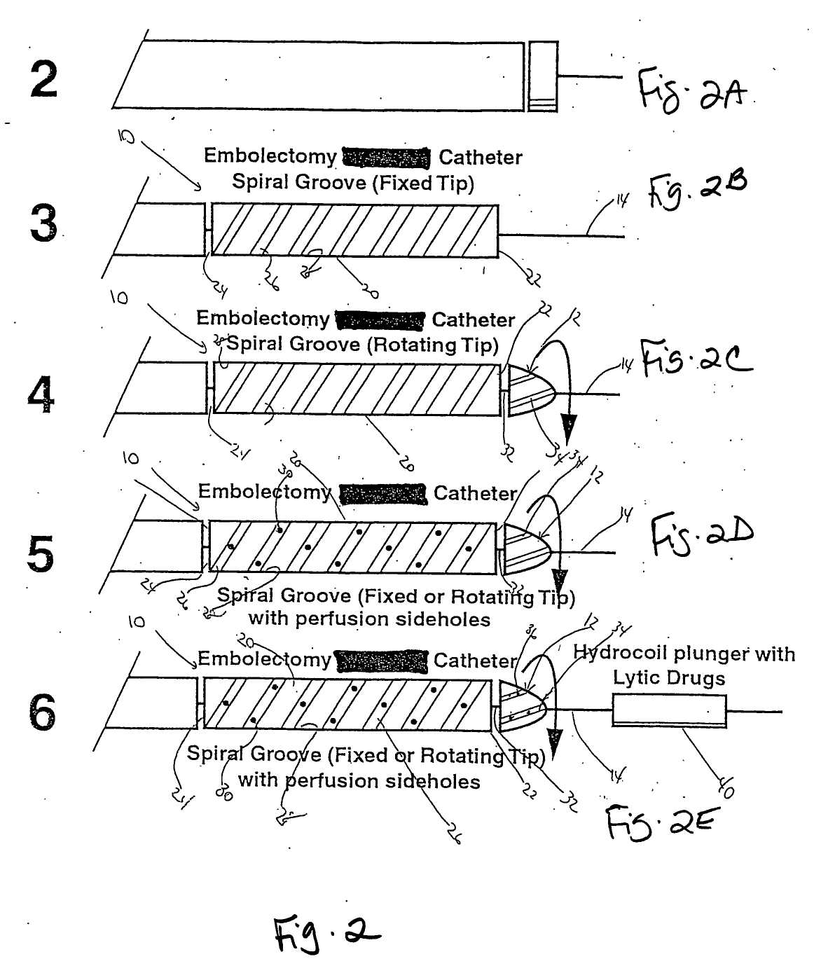

### DESCRIPTION OF THE INVENTION

The present invention provides an embolectomy catheter, generally shown at 10 in the figures, and method of using the same. The catheter 10 is very flexible and includes a tip 12 that can be rotating or fixed. The various parts of the catheter are made of materials known to those of skill in the art that are sufficient to perform the method of the present invention.

The term “guidewire” as used herein can be any guidewire 14 known to those of skill in the art to be useful in treating thrombi. Examples of such guidewires 14 are well known to those of skill in the art. Preferably, the guidewire 14 includes both straight 16 and corkscrew 18 portions, such that the corkscrew portions of the guidewire enable the catheter to be advanced toward the clot. The corkscrew portions function as threading about which the catheter is wound. The guidewire includes a distal end and a proximal end.

The term “hydrophilic material” as used herein is intended to include a polymer network that is capable of absorbing and retaining a significant quantity of water within its network. The preferred hydrophilic material is a hydrogel material. The water absorption causes the material to expand or swell to a generally predictable degree depending on the initial size and shape. The high water content, flexibility, lack of or negligible toxicity, and strength of the hydrogel material somewhat resemble that of natural body tissue. The hydrogel material can be produced in a process as described in U.S. Pat. No. 4,663,358 incorporated herein by reference.

The present invention provides an embolectomy catheter 10 for removing an embolus from a body artery or vein. The catheter 10 includes an elongated hollow lumen 20, having an insertion end 22 and an opposite end 24. The catheter lumen 20 is formed of a flexible and durable material. Examples of such materials include, but are not limited to, a polymeric material or other materials known to those of skill in the art.

The embolectomy catheter device 10 is an elongate, pliable clot penetrating catheter 10 that is advanceable, insertion end 22 first, through the clot or other obstructive matter (e.g., thrombus, thromboembolus, pieces of detached atherosclerotic plaque, foreign matter, etc.) that is to be removed.

The catheter 10 of the present invention includes grooves 26 about the exterior surface 28 thereof. Such grooves 26 can be spirally formed or helically formed about the exterior 28 of the catheter 10. The grooves 26 enable the catheter 10 to more effectively and efficiently break apart thrombi. The grooves 26 function in a manner similar to a drill bit. In other words, the grooves 26 provide the ability of the catheter 10 to both advance through the clot and to dissolve or break apart the clot by spirally penetrating and eventually breaking apart the obstruction.

The catheter 10 of the present invention can also include perfusion sideholes 30. The sideholes 30 provide the ability of the catheter 10 to introduce, at the location of the clot, liquids that are beneficial in breaking apart clots. The sideholes 30 are sized such that the liquid can be introduced through the lumen 20 and out of the sideholes 30 at the desired location. The size of the sideholes 30 can vary depending upon the liquid to be introduced and such sizing can be varied by those of skill in the art to affect the desired result.

The liquids that can be inserted can include, but are not limited to, saline and thrombolytic agents. The thrombolytic agents can be any clot dissolving agents known to those of skill in the art. Examples of thrombolytic agents include, but are not limited to, streptokinase, kabikinase, tPA activase, recombinant alteplase, anistreplase, recombinant reteplase, Anisoylated plasminogen-streptokinase activator complex, APSAC, tissue-type plasminogen activator (recombinant), t-PA, rt-PA, prourokinase, and urokinase.

The catheter device 10 of the present invention includes an elongate, pliable lumen 20 having a grooved tip 12 attached at an insertion end 22, as shown in the Figures. The grooved tip 12 can be either fixed or rotatable about a central axis of the lumen 20 of the catheter 10. Preferably, the tip 12 is affixed via a coupling joint 32, but it can be affixed in other manners known to those of skill in the art capable of rigidly affixing the tip 12.

The tip 12 of the present invention is preferably cone shaped. The rotating tip/cone head 12 mechanically breaks up the clot/thrombus. The cone shaped head 12 rotates on a corkscrew segment 18 of guidewire 14. The tip 12 is made of a material known to those of skill in the art that is sufficient to break up a clot. The tip 12 has grooves 34 such that the grooves 34 better dissolve or break apart the clot. Additionally, as with the catheter lumen 20, the tip 12 can include perfusion sideholes 36 for introducing liquid at the site of the clot.

A guidewire lumen 38 extends longitudinally through the entire length of the catheter 10 (i.e., an “over-the-wire” embodiment) or through only an insertion portion 22 of the catheter 10. In either of these embodiments of the catheter 10, the guidewire lumen 18 extends through the catheter such that the catheter can be advanced over a guidewire 14 that has previously been passed through the vessel-obstructing clot or other obstructive matter. Such arrangement of the guidewire lumen 38 additionally allows the embolectomy catheter 10 to be exchanged (e.g., removed and replaced with another embolectomy catheter 10 or another type of catheter) if such exchange should become necessary or desirable. This ability to allow the guidewire 14 to remain positioned through the offending clot or other obstructive matter can serve to ensure that the catheter 14 or its replacement can be re-advanced through the clot or other obstructive matter to its desired position.

A plunger 40 can be affixed at a proximal end of the guidewire. The plunger 40 is preferably formed of a soft hydrophilic material. Most preferably, the plunger 40 is formed of an expandable material, such that the plunger 40 can prevent distal migration of macerated fragments of the clot. Examples of such materials include, but are not limited to, hydrogels.

A contrast medium injection can also be injected through the sideholes 30. This enables the injection of radiographic contrast medium through the lumen 20 and out of the insertion end 22 of the catheter 10. In this regard, it is preferable that the outer diameter of the guidewire 14 be at least slightly less than the inner diameter of the lumen to permit some radiographic contrast medium to pass through the lumen and out of the distal end of the catheter even when the guidewire is positioned within the lumen. Also, radiographic contrast solutions (i.e., dyes) of minimal viscosity can be selected to enhance the ability of the contrast medium to pass through the lumen while the guidewire is positioned therewithin.

Initially the insertion end 22 of the catheter 10 is advanced through the clot or other obstructive matter. To assist the catheter 10 in passing through the clot or other obstructive matter, energy (e.g., radio-frequency energy, vibration, heat, etc) can be applied to the proximal strut(s) during their proximal retraction through the clot or other obstructive matter.

The catheter 10 is useful for cerebral vasculature, i.e. basilar artery stem and middle central artery or main stem of internal carotid artery. Acute thrombosis of cerebral vasculature by an embolus or thrombus is a major cause of acute CNS stroke. Currently designed embolectomy devices are rigid, bulky, and very expensive and they do not have control over distal migration of broken thrombi. Currently available thrombolytic agents do not consistently lyse the blood clots due to various different types of clot and their fibrin/platelet content. The catheter 10 of the present invention can prevent distal migration and can lyse blood clots.

Multiple roles played by the catheter of the present invention have a significant potential for a marketable medical device. Although primarily designed for central vasculature, the device can be safely utilized in other organs, e.g. coronary artery or limb vessels. The catheter for embolectomy in accordance with the present invention can also be used for treating, for example, other blood vessel such as esophageal varices, other aneurysms excluding cerebrovascle, e.g., aortic aneurysm. It can be also be used for treating disease, for example, and prosthesis method in lumen and ventor such as for prostheses of a removed portion after cutting a tissue such as cancer and tumor by surgical operation using in endoscope.

Throughout this application, various publications, including United States patents, are referenced by author and year, and patents, by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

What is claimed is:

1. A embolectomy catheter comprising a lumen including a grooved insertion end.

2. The catheter according to claim 1, wherein said catheter includes a grooved tip affixed to said insertion end.

3. The catheter according to claim 2, wherein said tip is a fixed tip.

4. The catheter according to claim 2, wherein said tip is rotating tip relative to said lumen.

5. The catheter according to claim 1, wherein said grooved tip is affixed via a coupling joint rigidly affixed at said insertion end of said catheter.

6. The catheter according to claim 1, wherein said grooved insertion end includes a spiral groove formed on an exterior surface of said lumen.

7. The catheter according to claim 1, further including perfusion sideholes extending through an exterior surface of said catheter.

8. The catheter according to claim 1, further including a plunger operably connected to said insertion end of said catheter for preventing migration of fragment of a thrombus.

9. The catheter according to claim 8, wherein said plunger is formed of a hydrophilic material.

10. The catheter according to claim 9, wherein said hydrophilic material is a hydrogel.

11. A method of treating a thrombus in an individual in need of treatment by:
inserting the catheter according to claim 1 into an individual at a location in need of treatment, and
rotating the catheter within the individual at the location in need of treatment, thereby breaking apart the thrombus.

12. The method according to claim 11, wherein said rotating step including spirally rotating the catheter to break apart the thrombus.

13. The method according to claim 11, further including the step of removing the broken fragments of the thrombus.

14. The method according to claim 11, further including administering thrombolytic agents to the thrombus prior to rotating the catheter.

15. The method according to claim 14, wherein said administering step includes administering the thrombolytic agents through perfusion sideholes within the catheter.

16. The method according to claim 11, further including preventing distal migration of fragment of the thrombus.

17. The method according to claim 16, wherein said preventing step includes inserting a plunger attached to the catheter, the plunger preventing distal migration.

18. The method according to claim 11, further including administering a thrombolytic agent through the catheter.

19. The method according to claim 16, wherein said administering step includes spraying the thrombolytic agent through sideholes within the catheter.
